(19) United States
(12) Patent Application Publication
(10) Pub. No.: US 2024/0390663 A1
(43) Pub. Date: Nov. 28, 2024

(54) PERCUTANEOUS DEVICE

(71) Applicant: The General Hospital Corporation, Boston, MA (US)

(72) Inventors: John Di Capua, Jr., Boston, MA (US); Avik Som, Boston, MA (US)

(73) Assignee: The General Hospital Corporation, Boston, MA (US)

(21) Appl. No.: 18/795,098

(22) Filed: Aug. 5, 2024

Related U.S. Application Data
(63) Continuation-in-part of application No. PCT/US2023/062049, filed on Feb. 6, 2023.
(60) Provisional application No. 63/306,932, filed on Feb. 4, 2022.

(51) Int. Cl. A61M 39/10; A61M 1/00; A61M 25/02; A61M 39/24

(52) U.S. Cl. CPC A61M 39/10; A61M 25/02; A61M 39/24; A61M 1/84; A61M 2025/0273; A61M 2039/1061; A61M 2039/1066; A61M 2205/0288

(57) ABSTRACT

Systems and methods for a coupling a percutaneous device. The system can include a first coupling that can be configured to connect to a first catheter extending through a percutaneous opening in a patient, a second coupling that can be configured to connect to a second catheter to fluidly connect to the first catheter, and a failure-release that can be configured to automatically disconnect the fluid connection between the first catheter and the second catheter upon the system receiving a force at or above a predetermined threshold to protect the percutaneous opening in the patient from the force.

### STATEMENT REGARDING FEDERALLY SPONSORED RESEARCH

Not Applicable.

### RELATED APPLICATIONS

This application is a continuation-in-part of international Application No. PCT/US2023/062049 filed Feb. 6, 2023, which claims priority to U.S. Provisional Patent Application No. 63/306,932 filed Feb. 4, 2022. The entirety of each is incorporated herein by reference.

### BACKGROUND

The disclosure relates generally to percutaneous systems, and more specifically, to systems and methods for mitigating issues related to percutaneous system dislodgement.

Percutaneous devices are well known in medical care and commonly placed to address a variety of conditions involving the need to drain a substance from an abdominal or thoracic cavity of a patient to an external environment (e.g., for draining fluids from treatment of an internal infection).

### BRIEF SUMMARY

The present disclosure provides systems and methods for a failure-release within a percutaneous system and also systems and methods for securing a percutaneous device to a patient.

In one configuration, a system can be configured to be coupled to a percutaneous device. The system can include a first coupling that can be configured to connect to a first catheter extending through a percutaneous opening in a patient. A second coupling can be configured to connect to a second catheter to fluidly connect to the first catheter. A failure-release can be configured to automatically disconnect the fluid connection between the first catheter and the second catheter upon the system receiving a force at or above a predetermined threshold to protect the percutaneous opening in the patient from the force.

The failure-release can include a set of magnets, including a first magnet attached to the first coupling and a second magnet attached to the second coupling. The first and second magnets can be removably coupled via a magnetic attraction therebetween. The predetermined threshold can be equal to a magnetic attraction threshold of the magnetic attraction between the first and second magnets. The first and second magnets can be ring magnets.

The system can further include a retainer configured to be secured to skin of the patient adjacent the insertion site and configured to retain the first catheter therein. The retainer can be sized to accommodate and configured to retain a portion of the first catheter formed in a coil. The retainer can include an arcuate surface around which the first catheter can be wrapped. The arcuate surface can be textured to increase surface friction between the first catheter and the arcuate surface. The retainer can include a plurality of notches about a peripheral wall thereof through which the first catheter can pass through.

The first coupling can include a one-way valve and the second coupling can include a stem configured to extend into the first coupling to open the one-way valve.

In another configuration, a system can be configured to be coupled to a percutaneous device and can include a failure-release operable between an engaged configuration and a disengaged configuration and a one-way valve. The failure-release and the one-way valve can be configured to be integrated with a catheter extending through a percutaneous opening in a patient. With the failure-release in the engaged configuration the one-way valve is in an open configuration and fluid flow is permitted through the catheter, the one-way valve, and out through the failure-release. With the failure-release in the disengaged configuration the one-way valve is in a closed configuration and fluid flow is prohibited through the catheter.

The failure-release can include a set of magnets, including a first magnet and a second magnet. With the failure-release in the engaged configuration, the first and second magnets are magnetically coupled. With the failure-release in the disengaged configuration, the first and second magnets are magnetically decoupled and spaced apart. The failure-release can include a stem coupled to the second magnet and the one-way valve can be coupled to the first magnet. With the failure-release in the engaged configuration, the stem urges the one-way valve out of a normally closed state, positioning the one-way valve in the open configuration. With the failure-release in the disengaged configuration, the stem is spaced from the one-way vale, the one-way valve is in the normally closed state, positioning the one-way valve in the closed configuration.

The system can further include a retainer configured to retain the catheter on the patient between the percutaneous opening and the failure-release. The retainer can include a cover and a base. The base can be configured to be adhered to the patient. The cover or the base can have an arcuate surface around which the catheter can be wrapped. The cover or the base can have a plurality of notches about a peripheral wall thereof through which the catheter can pass through.

In another configuration, a system can be configured to be used with a percutaneous device and can include a retainer including a base. The base can be configured to be secured to skin of the patient adjacent a percutaneous opening in a patient. The retainer can be configured to retain a catheter extending through the percutaneous opening.

The system can further include a first coupling configured to connect to a first catheter extending through a percutaneous opening in a patient. A second coupling can be configured to connect to a second catheter. The second coupling can be configured to be coupled with the first coupling to fluidly connect to the first catheter and the second catheter. A failure-release can be configured to automatically disconnect the fluid connection between the first catheter and the second catheter upon the system receiving a force above a predetermined threshold to protect the percutaneous opening in the patient from the force.

### BRIEF DESCRIPTION OF DRAWINGS

The disclosure will be better understood and features, aspects, and advantages other than those set forth above will become apparent when consideration is given to the following detailed description thereof. Such detailed description makes reference to the following drawings which may not be drawn to scale.

FIG. 1 is an illustration of a generic percutaneous system installed on a patient.

FIG. 2 is a top plan view of a percutaneous system according to the present disclosure.

FIG. 3 is a top plan view of a failure-release coupling of the percutaneous system of FIG. 2 shown in an engaged configuration according to the present disclosure.

FIG. 4 is a side elevation view of the failure-release coupling shown in a disengaged configuration according to the present disclosure.

FIG. 5 is an isometric view of a first coupling of the failure-release coupling of FIG. 3 according to the present disclosure.

FIG. 6 is an isometric view of the first coupling of FIG. 5 with a magnet and a one-way valve according to the present disclosure.

FIG. 7 is an isometric view of the first coupling of FIG. 6.

FIG. 8 is an isometric view of a one-way valve according to the present disclosure.

FIG. 9 is a side elevation view of the one-way valve of FIG. 8.

FIG. 10 is an isometric view of a one-way valve according to another example of the present disclosure.

FIG. 11 is an isometric view of a second coupling of the failure-release coupling of FIG. 3 according to the preset disclosure.

FIG. 12 is an isometric view of the second coupling of FIG. 11 with a magnet according to the present disclosure.

FIG. 13 is an isometric view of the second coupling of FIG. 12.

FIG. 14 is an exploded isometric view of a retainer of the percutaneous system according to the present disclosure.

FIG. 15 is an isometric view of the retainer of FIG. 14.

FIG. 16 is a side elevation view of another failure-release coupling shown in a disengaged configuration according to the present disclosure.

FIG. 17 is an isometric view of a first coupling of the failure-release coupling of FIG. 16 according to the present disclosure.

FIG. 18 is an isomeric view of the first coupling of FIG. 17.

FIG. 19 is an isometric view of a second coupling of the failure-release coupling of FIG. 16 according to the present disclosure.

FIG. 20 is an isometric view of the second coupling of FIG. 19.

FIG. 21 is a top plan partially exploded view of a percutaneous system according to the present disclosure.

FIG. 22 is an isometric view of a percutaneous system in an open configuration according to the present disclosure.

FIG. 23 is an isometric view of the percutaneous system of FIG. 22 in a closed configuration according to the present disclosure.

FIG. 24 is an isometric view of a retainer of the percutaneous system according to another example of the present disclosure.

FIG. 25 is an isometric view of another configuration of the failure-release couplings in a disengaged configuration according to another example of the present disclosure.

FIG. 26A is a cross-sectional view of the failure-release couplings of FIG. 25 according to the present disclosure.

26B is another cross-sectional view of the failure-release couplings of FIG. 25 according to the present disclosure.

FIG. 27 is an isometric view of the failure-release couplings of FIG. 25 in an engaged configuration according to the present disclosure;

FIG. 28 is a cross-sectional view of the failure release couplings of FIG. 27 in an engaged configuration according to the present disclosure.

### DETAILED DESCRIPTION

It is to be understood that the present disclosure is not limited in its application to the details of construction and the arrangement of components set forth in the following description or illustrated in the following drawings. The systems and methods described herein are capable of other configurations and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

Also as used herein, unless otherwise specified or limited, directional terms are presented only with regard to the particular configuration and perspective described. For example, reference to features or directions as “horizontal,” “vertical,” “front,” “rear,” “left,” “right,” and so on are generally made with reference to a particular figure or example and are not necessarily indicative of an absolute orientation or direction. However, relative directional terms for a particular configuration may generally apply to alternative orientations of that configuration. For example, “front” and “rear” directions or features (or “right” and “left” directions or features, and so on) may be generally understood to indicate relatively opposite directions or features.

As used herein in the context of activities or engagement of components, unless otherwise specified or limited, “manual” refers to the use of human hands. In some cases, “manual” engagement or activity can include direct manual engagement or activity: i.e., engagement or activity directly conducted by a user's hands (e.g., a user grasping or manipulating an object by hand). In some cases, “manual” engagement or activity can include engagement or activity via a non-powered hand tool (e.g., pliers).

The following discussion is presented to enable a person skilled in the art to make and use configurations of the present disclosure. Various modifications to the illustrated configurations will be readily apparent to those skilled in the art, and the generic principles herein can be applied to other configurations and applications without departing from configurations of the present disclosure. Thus, configurations of the disclosure are not intended to be limited to configurations shown, but are to be accorded the widest scope consistent with the principles and features disclosed herein. The following detailed description is to be read with reference to the figures, in which like elements in different figures have like reference numerals. The figures, which are not necessarily to scale, depict selected configurations and are not intended to limit the scope of configurations of the disclosure. Skilled artisans will recognize the examples provided herein have many useful alternatives and fall within the scope of configurations of the disclosure.

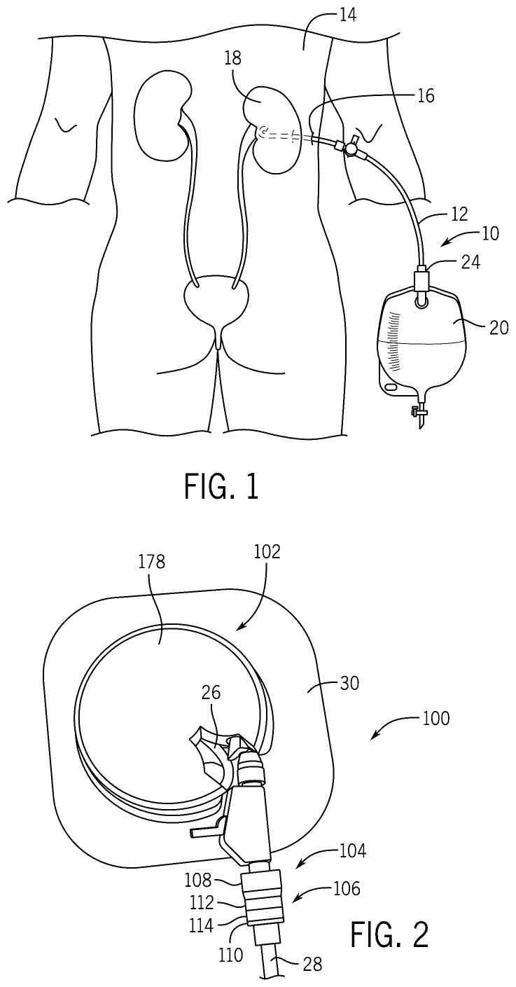

As noted above, in some instances, it may be necessary to drain fluid, such a substance from an abdominal or thoracic cavity of a patient to an external environment. An example of a percutaneous system 10 as commonly known is shown in FIG. 1. In the practice of percutaneous, a percutaneous catheter 12 is inserted through the patient's skin 14 at an insertion site 16 and into the cavity 18 (e.g., a kidney) to be drained to a drainage bag 20 through a drainage bag tubing 22. Typically, a Luer lock connection 24 is used to attach the percutaneous catheter 12 to the drainage bag tubing 22. This type of attachment is very secure, with the percutaneous catheter 12 secured to the drainage bag tubing 22 in a contiguous piece.

Patients are often discharged home with such percutaneous systems, which are left in place for several days to weeks to allow the substance, typically fluid or air, to drain outside the body. It is estimated that upward of 3.2 million nonvascular percutaneous devices (e.g., drains, enteral access, etc.) and 11.2 million vascular devices are placed in patients (e.g., in the chest or abdomen) in the United States annually. A non-exhaustive list of potential indications for the placement of a percutaneous device into the abdomen (accounting for an estimated 100 thousand annual placements in the US) includes: drainage of abscess(es) associated with: complicated appendicitis, complicated diverticulitis, necrotizing pancreatitis, or inflammatory bowel disease (IBD), as well as post-operative placement. The following is a non-exhaustive list of potential indications for the placement of a percutaneous drain device into the chest: pneumothorax, chest trauma, chylothorax, hemothorax, pleural effusion, complicated pneumonia-related effusion, empyema, or post-operative care.

While existing percutaneous devices, such as the system 10 shown in FIG. 1, are recognized to be essential and effective in treating the conditions noted above, these devices are often associated with their own complications that can have a significant negative impact on patient outcomes. One of the most common potential adverse events associated with percutaneous drain placement is accidental dislodgement of the drain device, which occurs in as many as between 4.5% to 12.8% of patients who use percutaneous drains outside the hospital environment and between 28% to 42% for thoracic drains alone. Partial or complete dislodgement is most often due to an unexpected force on the external drain tubing or drainage bag. The complications of such an event vary depending on the type of catheter but typically arise from the removal of the device itself (damage to tissues during catheter removal), interruption in drainage of the intended target (e.g., for performing necessary medical therapy), and risk related to additional imaging and procedures required for reinsertion of the catheter. For example, the drainage bag tubing 22 of the system 10 could get stuck on a piece of furniture when the patient moves or the drainage bag 20 could accidentally drop. Any disruption to the internal portion of the catheter 12, even if not fully removed from the cavity 18, can displace the percutaneous catheter 12 from the targeted cavity 18 and render the catheter 12 nonfunctional. Once a drainage catheter is dislodged, patients must present to the emergency room for evaluation which typically includes laboratory testing, imaging, and if necessary, hospital admission and replacement of a new drain. Beyond dislodgement, patients with percutaneous devices often experience substantial discomfort related to irritation of the skin by the catheter, which persists for as long as the catheter is in place, indefinitely in some cases for a subset of patients who have chronic infections or cancer.

A percutaneous system according to the disclosure can create an intentional failure point via a failure-release mechanism along the external portion of the drainage catheter that allows the tubing to dissociate at a fixed location following the application of an inappropriate external force. Other aspects of the percutaneous system can include securement of the external portion of the drainage catheter to the skin of a patient. The proposed system can reduce the number of accidental catheter removals, which in turn will reduce interruptions in patient therapy, reduce delay in necessary patient care, reduce patient risk due to avoidable imaging and repeat procedures for catheter reinsertion, and greatly improve the patient experience. According to some aspects, the system can be compatible with percutaneous catheters (e.g., pig-tail drainage catheters) and can be incorporated with catheters using a Luer lock connection (e.g., chest tubes, central lines, peripheral IVs, etc.), ENFit® fittings, or slip-tip connections, for example.

Configurations of the present disclosure can address the issues stated above and other risks associated with percutaneous systems. Generally, configurations of the disclosure provide a percutaneous device less susceptible to dislodgement. The percutaneous device can be configured to break apart at a point along the catheter when the catheter experiences a force at or greater than a threshold force. The threshold force can be a predetermined force that is greater than the weight of a drainage bag and less than the adhering strength of an adhesive adhering the catheter to skin of a patient. The maximum threshold force can also be determined with respect to a reference force. For example, the predetermined force can be a fraction of the force (reference force) required to overcome the adhesion of a retainer that attaches the catheter to a patient's skin). The break-apart point along the catheter can be configured with a failure-release that can be reassembled by a patient after a failure event. In some examples, the failure-release can include a set of magnets on a set of couplings. The magnets decoupling from magnetic attraction allowing the couplings to separate during a failure event. In some examples, the failure-release can be a ball and socket connection or other type of similar separable and reconnectable joint such as, for example, a friction-fit connection, a snap-fit connection, etc.

Additionally, a one-way valve can be included in the failure-release to cut flow of fluid out or flow of air into the catheter after a failure event. For example, a first coupling can include a stem that deforms the one-way valve out of a normally-closed state in a second coupling to allow fluid to flow thereby when in an engaged configuration, and when the first and second couplings are separated in a disengaged configuration after a failure event, the stem is removed from contact with the one-way valve, allowing the one-way valve to return to the normally-closed state and prevent flow through the catheter.

Further, some configurations of the present disclosure of a percutaneous device can include a retainer that can be adhered adjacent an insertion site of a catheter into a patient. In some examples, the retainer can retain a coiled portion of the catheter. The coiled portion can be wrapped around an arcuate surface within the retainer and exit through an opening in a peripheral wall thereof. Wrapping the catheter within the retainer effectively secures the catheter to the patient. In some examples, the retainer can be a locking clip adhered to a patient's skin adjacent an insertion site and configured to retain the catheter within the locking clip.

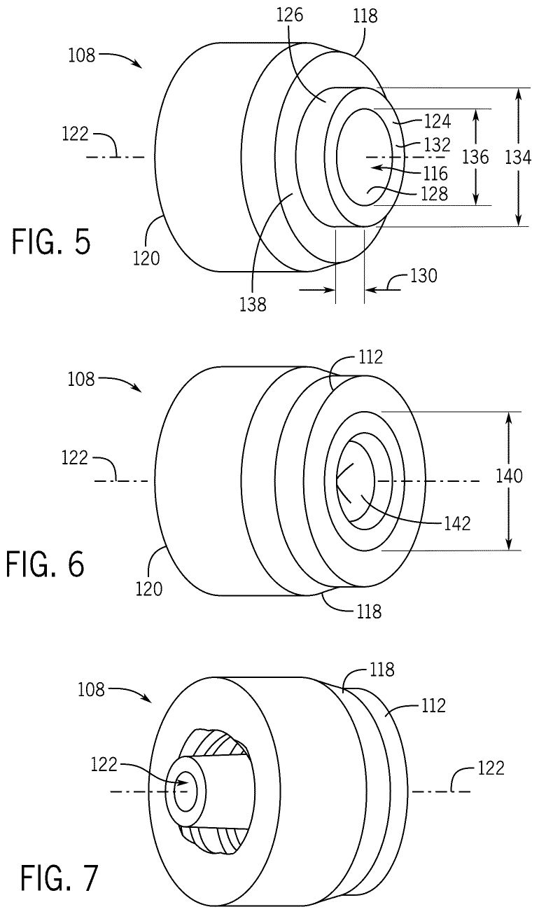

FIGS. 2-14 illustrate a percutaneous system 100 and elements thereof according to an example of the present disclosure. With reference to FIG. 1, the percutaneous system 100 can include a retainer 102 and a failure-release coupling 104, including a failure-release 106, a first coupling 108, and a second coupling 110. As shown, a first catheter 26 extends into the skin at an insertion site (e.g., the insertion site 16 shown in FIG. 1) on one end, is secured to the retainer 102, and is attached to the failure-release coupling 104 at the first coupling 108 at the other end. A second catheter 28 is attached to the failure-release coupling 104 at the second coupling 110 and terminates at a drainage bag (e.g., the drainage bag 22 shown in FIG. 1). Fluid being drained from the patient can thus flow out of the patient through the first catheter 26 at the insertion site, through the first and second couplings 108, 110 and further out through the second catheter 28 into the drainage bag 22 or other receptacle.

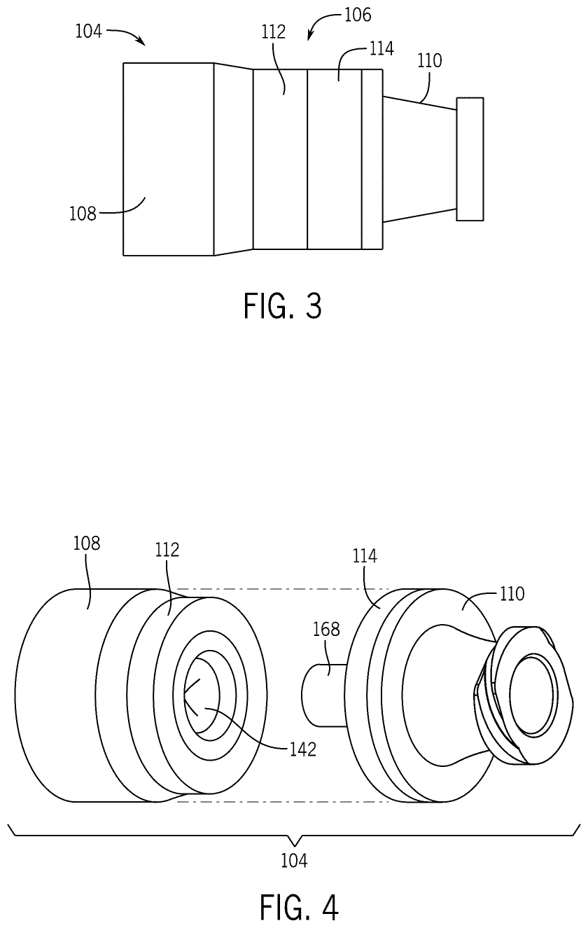

Continuing, FIGS. 3 and 4 show the failure-release coupling 104 in isolation. The first and second couplings 108, 110 are shown with the failure-release 106 in an engaged configuration in FIG. 3 and in a disengaged configuration in FIG. 4. The failure-release 106 is configured to break apart the failure-release coupling 104 and separate the first and second couplings 108, 110 when the second catheter 28 experiences a force at or greater than a threshold force. As illustrated in FIGS. 3 and 4, the failure-release 106 includes a set of magnets, including a first magnet 112 attached to the first coupling 108 and a second magnet 114 attached to the second coupling 110. The first and second magnets 112, 114 are arranged to be attracted to each other and magnetically couple the first and second couplings 108, 110 when the failure-release 106 is in the engaged configuration.

FIGS. 4 through 7 provide further illustration of the first coupling 108. The first coupling 108 has a passageway 116 extending from a first end 118 through a second end 120 along a first coupling axis 122. The first end 118 has a protrusion 124 of generally cylindrical geometry with an outer surface 126 and an inner surface 128 extending in parallel a first distance, defining a first length 130, concentrically along the first coupling axis 122, and a facing surface 132 extending between the outer and inner surfaces 126, 128 and perpendicular to the first coupling axis 122. The outer surface 126 defines an outer diameter 134 of the protrusion 124 and the inner surface 128 defines an inner diameter 136 thereof. Further, the protrusion 124 defines a radial surface 138 on the first end 118 of the first coupling 108 adjacent the protrusion 124. The second end 120 of the first coupling 108 can be formed as a half of a Luer lock connector configured to be connected to a corresponding half of a Luer lock connector attached to the first catheter 26. Here, the second end 120 is provided as a female half of a Luer lock connector. However, the second end 120 could also be formed as a male half of a Luer lock connector.

As shown in FIG. 6, the first magnet 112 can be a ring magnet. The first magnet 112 thus has an inner diameter 140, which is approximately the same dimension as the outer diameter 134 of the protrusion 124, on which the first magnet 112 is placed. The first magnet 112 can be positioned abutting the radial surface 138 of the first coupling 108 to fully seat the first magnet 112 on the protrusion 124.

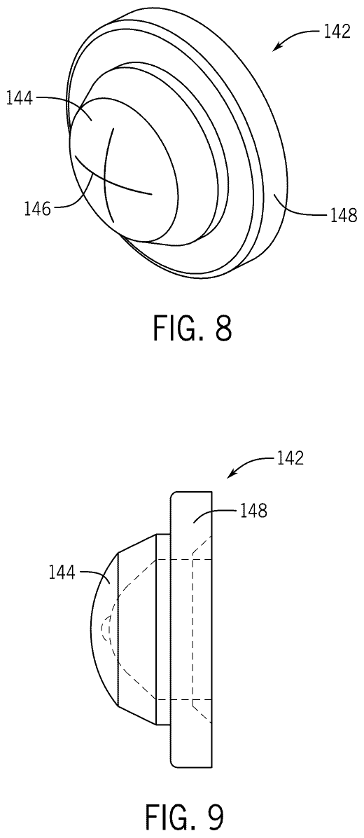

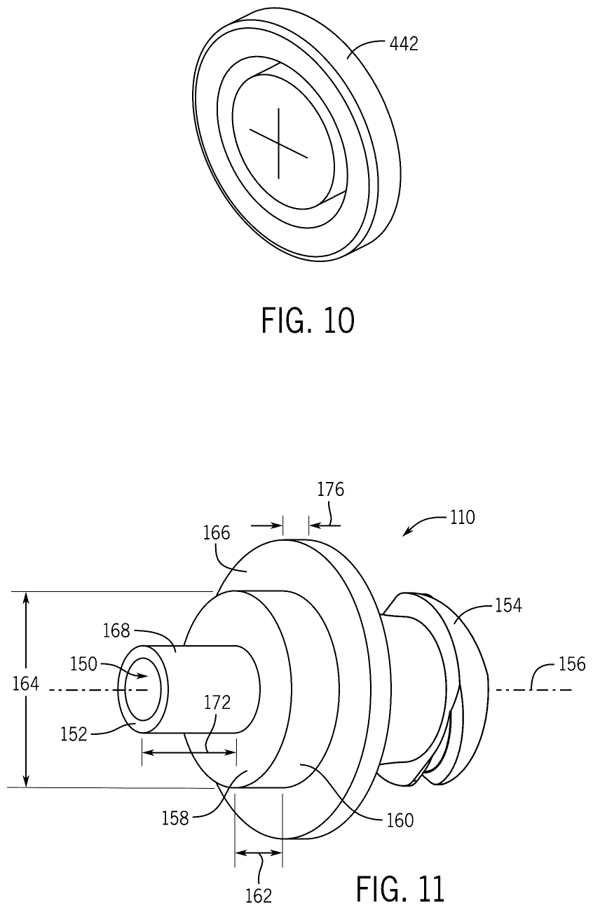

Continuing, a one-way valve 142 can be provided at the first end 118 of the first coupling 108 as illustrated in FIG. 6. An example of the one-way valve 142 is shown in greater detail in FIGS. 8 and 9. The one-way valve 142 can be a cross-slit valve including a dome 144 with a set of slits 146 and a flange 148 radially extending from the base of the dome 144. The one-way valve 142 is sized and configured for the dome 144 to be received within the passageway 116 at the first end 118 of the first coupling 108, with the flange 148 abutting the facing surface 132. In other examples, other types of one-way valves may be used, for example, a duckbill valve or another type of flexible one-way valve, including other forms of cross-slit valves could be used. For example, as provided in FIG. 10, a one-way valve 442 according to the disclosure could be a cross-slit valve having a disc-like shape.

FIGS. 11 through 13 provide further illustration of the second coupling 110. The second coupling 110 has a passageway 150 extending from a first end 152 through a second end 154 along a second coupling axis 156. The first end 152 has a protrusion 158 of generally cylindrical geometry with an outer surface 160 extending a second distance, defining a second length 162, concentrically along the second coupling axis 156 and has an outer diameter 164. The protrusion 158 further defines a radial surface 166 adjacent the protrusion 158. The first end 152 also has a stem 168 extending from the protrusion 158 a third distance, defining a third length 170, concentrically along the second coupling axis 156. The second end 154 of the second coupling 110 can also be formed as a half of a Luer lock connector configured to be attached to a corresponding Luer lock half attached to the second catheter 28. Here, the second end 154 is provided as a female half of a Luer lock connector, which corresponds to the male half of the second end 120 of the first coupling 108. However, the second end 154 could be formed as a male half of a Luer lock connector.

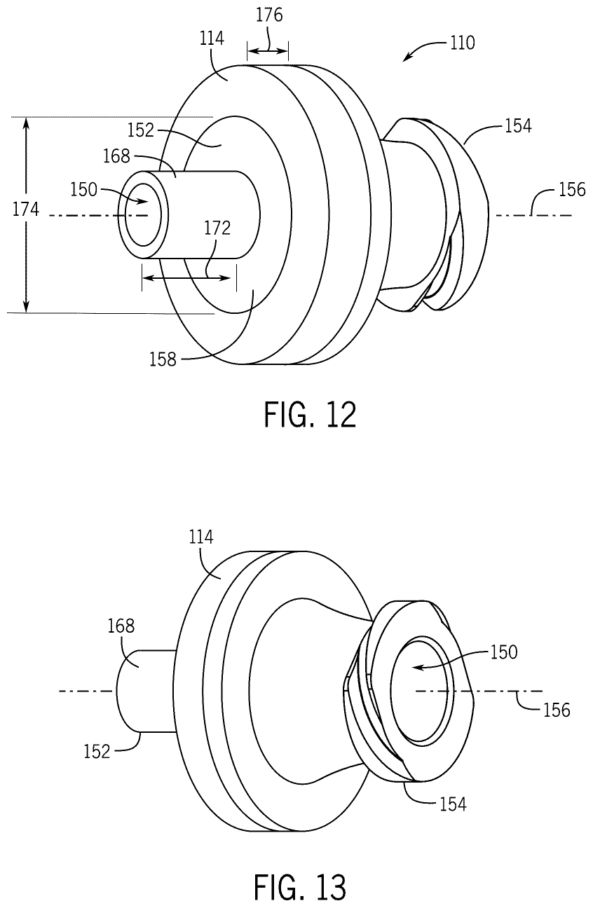

As shown in FIG. 12, the second magnet 114 can also be a ring magnet. The second magnet 114 has an inner diameter 172, which is approximately the same dimension as the outer diameter 164 of the protrusion 158 of the second coupling 110, on which the second magnet 114 is placed. The second magnet also has a thickness 174. The thickness 174 can be approximately equal to the second length 162 of the protrusion 158. The second magnet 114 can be positioned abutting the radial surface 166 of the second coupling 110 to fully seat the second magnet 114 on the protrusion 158.

As stated previously, the failure-release 106 of the failure-release coupling 104 can be configured to allow the separation of the first and second couplings 108, 110 from an engaged configuration (shown in FIG. 3) to a disengaged configuration (shown in FIG. 4) when the second catheter 28 experiences a force at or greater than a threshold force (i.e., a failure event). With the failure-release 106 in the disengaged position, the first and second magnets 112, 114 are magnetically decoupled and spaced apart. Further, the one-way valve 142 is normally-closed, whereby the dome 144 does not permit fluid to flow in a first direction, out from the patient, through the first catheter 26, and out through the first coupling 108. The dome 144 is also configured to prevent surrounding air at atmospheric pressure from entering into the first coupling 108 in a second direction, opposite the first direction, and through the first catheter 26 to the patient.

With the failure-release coupling 104 in the engaged configuration, the first and second magnets 112, 114 of the failure-release 106 are magnetically coupled. The stem 168 of the second coupling 110 is positioned at least partially within the passageway 116 of the first coupling 108 and through the dome 144 via the set of slits 146, thereby forcibly opening the one-way valve in the second direction to allow fluid to drain from the patient, through the first catheter 26, through the passageways 116, 150 of the first and second couplings 108, 110, respectively, through the second catheter 28, and to a drainage bag (e.g., the drainage bag 20 shown in FIG. 1). Therefore, during a failure event, the second catheter 28 experiences a force at or greater than a threshold force, decoupling the first and second magnets 112, 114 and separating the first and second couplings 108, 110, which removes the stem 168 from the dome 144 allowing the dome 144 to return to the normally-closed state. In the normally-closed state, the dome 144 prevents fluid from draining out of the first coupling 108 and creating a mess on or around the patient. The dome 144 also prevents air at ambient pressure from entering through the first coupling 108, which can prevent complications, such as, for example, pneumothorax from occurring in chest applications.

It is further contemplated that in some examples of the disclosed system, a one-way valve, like the one-way valve 142 in the first coupling 108, can also be provided within the second coupling 110 configured to allow flow though the second catheter 28 but preventing fluid backflow out through the second coupling 110 when separated from the first coupling 108. In other examples, other types of one-way valves may be used, for example, a duckbill valve, an umbrella valve, or another type of flexible one-way valve, including other forms of cross-slit valves could be used.

The threshold amount of force required to invoke a failure event is a predetermined value. For example, it is contemplated that the minimum amount of force should be greater than the amount of force applied to the second catheter 28 from the drainage bag (e.g., when full) but not greater than the amount of force needed to overcome the adhesion of the retainer to the patient's skin. In some examples, the predetermined threshold force is in the range of about 3N to about 65N. In some examples, the threshold force is in the range of about 10N to about 45N. In some examples, the predetermined threshold force is in the range of about 5N to about 20N. In some examples, the predetermined threshold force is in the range of about 10N to about 20N. In some examples, the predetermined threshold force is about 15N. In some examples, the threshold force can be modified by using first and second magnets 112, 114 having stronger or weaker magnetic attraction. It is contemplated that the first and second magnets 112, 114 can be selected or changed by an installer in the hospital at the time of installation of the system 100 or by a patient after being released for adapting to the individual circumstances of the installation.

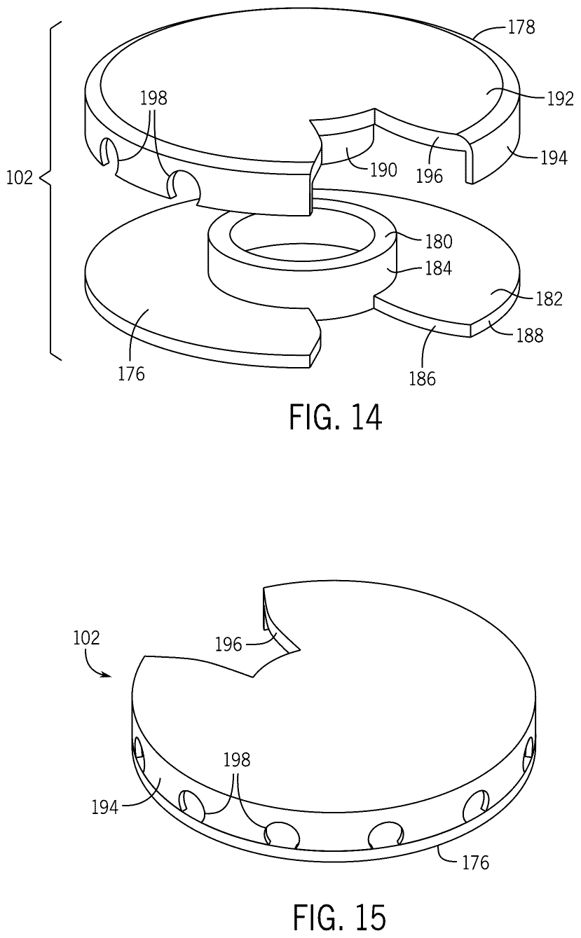

FIGS. 14 and 15 show the retainer 102 in isolation. The retainer 102 includes a base 176 and a cover 178 coupled to the base 178. In some examples, the base 176 and the cover 178 can be formed as a unitary, one-piece, unit. In some examples, the base 176 and the cover 178 can be attachable to each other via adhesive, friction fit, snap fit, or threaded fit, for example. The base 176 includes a collar 180 with a base flange 182 extending radially outward therefrom. The collar 180 has an arcuate surface 184 extending therearound. In some examples, a texture can be applied to or integrally formed within the arcuate surface 184 to increase the surface friction thereof. In some examples, the base 176 and the collar 180 can be formed from silicone, which will have an increased surface friction with a catheter because of the material characteristics of silicone. In some examples, a coating can be applied to the arcuate surface 184 to alter an otherwise smooth surface, such as, for example, a speckled or ribbed coating. The base flange 182 has a base opening 186 extending from the collar 180 to a peripheral edge 188 of the base flange 182. In some examples, the base 176 can be formed from a flexible material to allow the base 176 to conform to the contours of the location on the patient on which the retainer is affixed. For example, as mentioned above, in some examples the base 176 can be formed from silicone.

Continuing, the cover 180 of the retainer 102 as shown is configured to be attachable to the base 178. The cover 180 includes a plug 190 configured to be received within the collar 180 of the base 178 and a cover flange 192 extending radially outward therefrom. A peripheral wall 194 extends perpendicularly from and along the outer edge of the cover flange 192 and in the same direction as the plug 190. The cover flange 192 has a cover opening 196 extending from the plug 190 to and through the peripheral wall 194. The cover opening 196 can be sized and shaped similarly to the base opening 186, and when the base 176 and the cover 178 are combined, the cover opening 196 can be aligned with the base opening 186. Additionally, the peripheral wall 194 can include a plurality of notches 198 spaced there along.

It is contemplated that certain features of the base 176 could be included on the cover 178 and certain features of the cover 178 could be include on the base 176. For example, a base according to this disclosure could include a peripheral wall extending from a base flange and could include a plug receivable within a collar of a cover.

Installation of the retainer 102 can be accomplished by adhering the base 176 adjacent the insertion site (e.g., the insertion site 16 shown in FIG. 1). A flexible adhesive pad 30 can be included on the base 176 for providing a conforming and comfortable attachment to a patient. The first catheter 26 enters the retainer 102 at the opening 184 of the base 176 and is wrapped around the arcuate surface 184 of the collar 180. The plug 190 of the cover 178 is received within the collar 180 of the base 176 and the first catheter 26 can exit through any of the plurality of notches 198 or the cover opening 196 in the peripheral wall 194 of the cover 178. In some examples, the adhesive pad 30 can be imbued with medication to provide a controlled release of the medication to an area surrounding and including the insertion site 16.

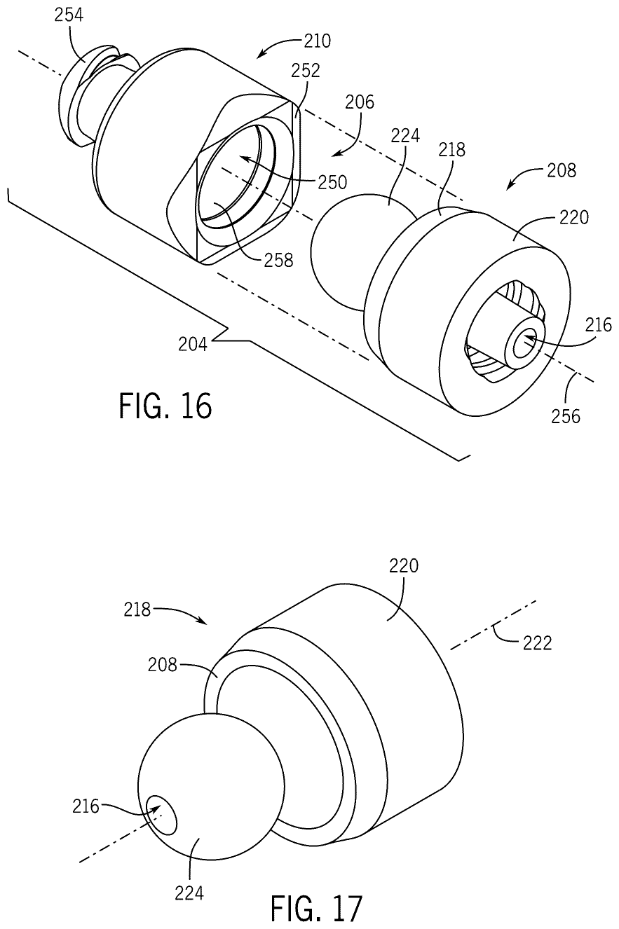

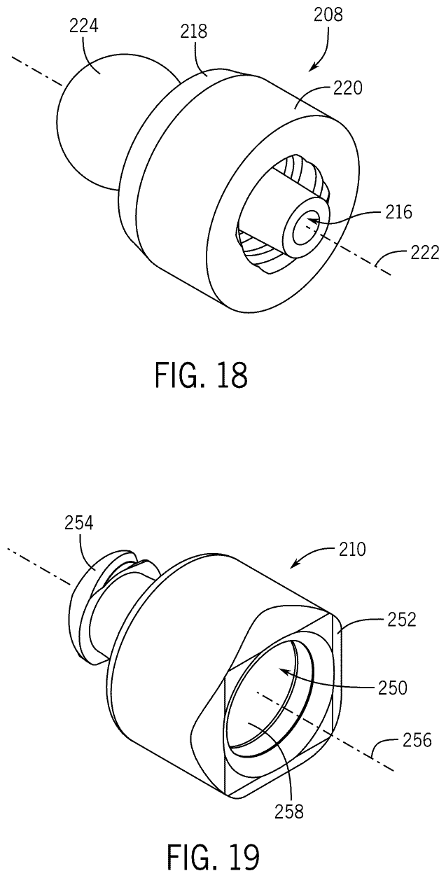

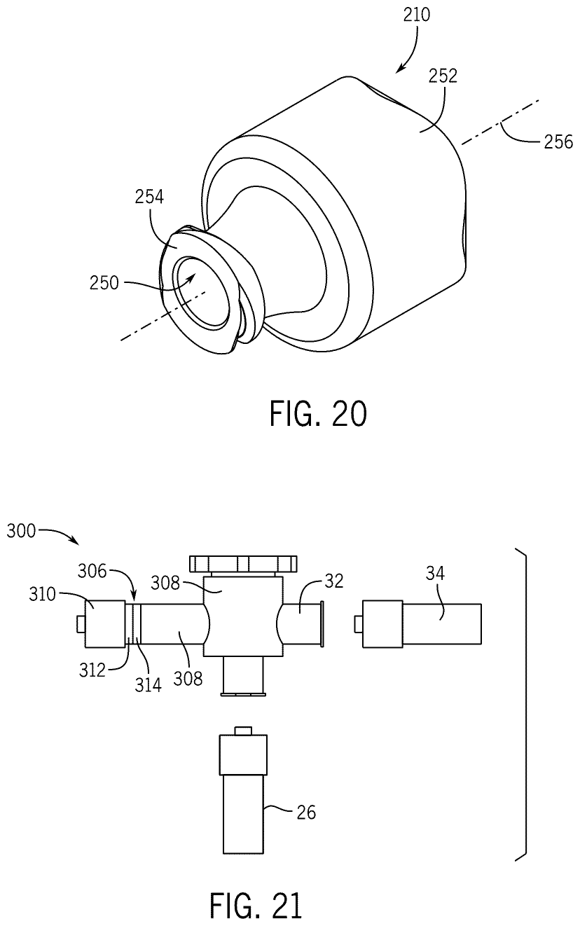

FIGS. 16 through 20 illustrate another example of a failure-release coupling 204 according to the disclosure, as also can be configured to transition from an engaged configuration to a disengaged configuration upon a failure event. In many aspects, the failure-release coupling 204 is similar to the failure-release coupling 104 described above and similar numbering in the 200 series is used for the failure-release coupling 204. For example, the failure-release coupling 204 includes a first coupling 208 (shown in isolation in FIGS. 17 and 18) with a passageway 216 extending along a first coupling axis 222 from a first end 218 to a second end 220. Further, the second end 218 is provided as a half of a Luer lock connector, here shown as a female half. Additionally, the failure-release 204 includes a second coupling 210 (shown in isolation in FIGS. 19 and 20) with a passageway 250 extending along a second coupling axis 256 from a first end 252 to a second end 254. The second end 254 is also provided as a half of a Luer lock connector, here shown as the male half.

In some aspects, however, the failure-release couplings 104, 204 differ from each other. For example, the first end 218 of the first coupling 208 has a protrusion 224 of generally spherical geometry, and the first end 252 of the second coupling 210 has a socket 258 configured to receive the protrusion 224 therein. The combination of the protrusion 224 and the socket 258 form a failure release 206. Thus, the protrusion 224 will be released from the socket 258 during a failure event. A one-way valve, similar or identical to the types of one-way valves discussed above with respect to the failure-release 104, can be provided within the socket 258 of the second coupling 210. The one-way valve can be configured to be urged open upon the insertion of the protrusion 224 of the first coupling 208 to allow flow through the failure-release coupling 204 and to prevent flow by closing upon removal of the protrusion 224.

In many cases, the system 100 will be worn by the patient for as long as required, including the remaining lifetime of the patient, and the patient or home health care provider will likely interface with the system 100 on a daily basis (regular drain maintenance with saline flushing as per standard clinical protocol). Therefore, it is contemplated that another example of the system 300 can further include a three-way valve 316 as shown in FIG. 21. In many aspects, the system 300 is similar to the system 100 described above and similar numbering in the 300 series is used for the system 300. For example, the system 300 includes a failure-release coupling 304 with a failure-release 306, a first coupling 308, and a second coupling 310. Additionally, the failure-release 306 includes first and second magnets 312, 314.

In some aspects, however, the systems 100, 300 differ from each other. For example, the first coupling 308 is integrally formed within the three-way valve 316. Further, the first catheter 26 is removably coupled to the three-way valve 316 and a flush port 32 is also attached to the three-way valve 316. In use, the three-way valve 316 is operable between an open flow configuration in which fluid flow through the three-way valve 316 is permitted from the first catheter 26, through the three-way valve 316, and out through at least the first coupling 308, a closed flow configuration in which fluid flow through the three-way valve 316 is prohibited, and a flushing configuration in which fluid flow of a flushing fluid is permitted from the a flush line 34 into the flush port 32, through the three-way valve 316, and out through at least the first coupling 308. Additionally, the first catheter 26 and the flushing line 34 can be connected to the three-way valve 316 via Luer lock connectors.

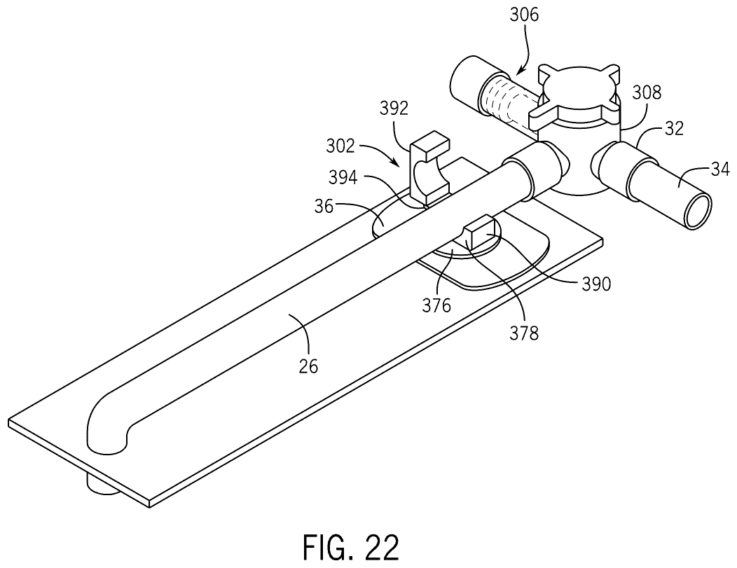

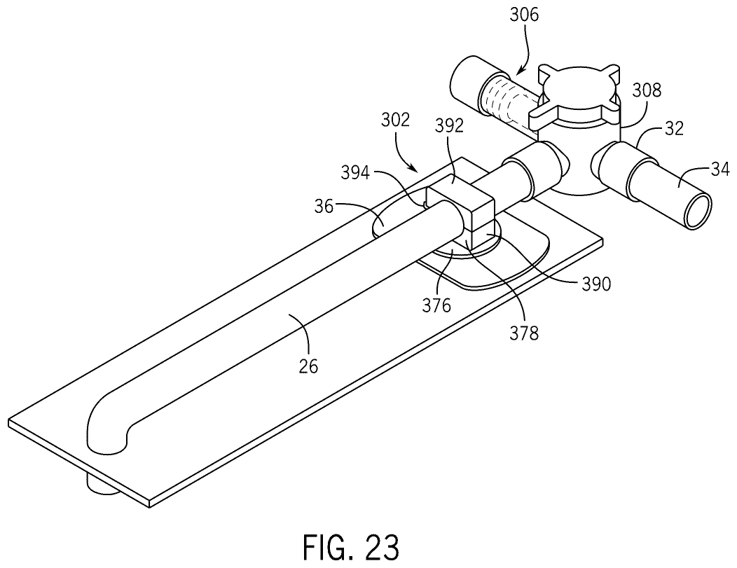

FIGS. 22 and 23 illustrate another example of a retainer 302 according to the present disclosure shown as part of the system 300 with the three-way valve 316 shown in FIG. 21. It is contemplated, however, that the retainer 302 could also be used to secure a catheter to a patient in other applications, such as, for example, securing the catheter 12 of the percutaneous system 10 shown in FIG. 1 to a patient. The retainer 302 can include a base 376 and a clip 378. Similar to the retainer 202, the retainer 302 can include a flexible adhesive pad 36 that adheres the retainer 302 to the skin of a patient adjacent an insertion site. The clip 378 is operable between an unlocked and open configuration (shown in FIG. 22) and a locked and closed configuration (shown in FIG. 23). As shown, the clip 378 can have a bottom portion 390 and a top portion 392 hingedly attached (e.g., via a living hinge 394) to the bottom portion 390. The free ends of the bottom and top portions 390, 392 opposite the hinge can include a clasping feature to secure the clip 378 in the closed configuration. In some examples, the bottom and top portions can be secured using clasping features on both ends.

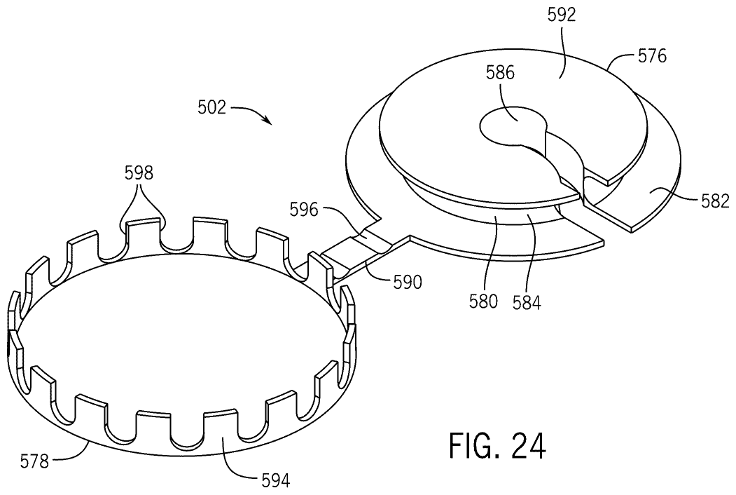

Another example of a retainer 502 according to the present disclosure is shown in FIG. 24. In many aspects, the retainer 502 is similar to the retainer 102 described above and similar numbering in the 500 series is used for the retainer 502. For example, the retainer 502 has a base 576 including a collar 580 with an arcuate surface 584 and a base 576 with a base flange 582 and a base opening 586. The retainer 502 also has a cover 578 including a peripheral wall 594 with a plurality of notches 598. The retainer 502 is also configured to receive and retain a catheter coiled therein to secure the catheter to a patient.

In some aspects, however, the retainers 102, 502 differ from each other. For example, the base flange 582 of the base 576 is a lower based flange extending radially outward from the bottom of the collar 580. The base 576 further includes an upper base flange 592 extending radially outward from the top of the collar 580. Additionally, the base opening 586 extends into the center of the base 576 and defines a curved pathway along which a catheter can be placed. Further, the base 576 and the cover 578 are coupled via a flexible tether 590, which can include at least one living hinge 596 to aid in bending the tether 590. Thus, in use, the base 576 can be secured to a patient and a catheter can enter the base 576 at the base opening 586 and wrapped around the arcuate surface 584. Then the cover 578 can be placed over the base 576 with the catheter exiting one of the plurality notches 598.

In some implementations, devices or systems disclosed herein can be utilized or installed using methods embodying aspects of the invention. Correspondingly, description herein of particular features or capabilities of a device or system is generally intended to inherently include disclosure of a method of using such features for intended purposes and of implementing such capabilities. Similarly, express discussion of any method of using a particular device or system, unless otherwise indicated or limited, is intended to inherently include disclosure, as embodiments of the invention, of the utilized features and implemented capabilities of such device or system.

For example, a method can include installing a catheter in a patient at an installation site, adhering a retainer to skin of the patient adjacent the installation site, securing the catheter to the retainer, and attaching a failure-release coupling to the catheter adjacent the retainer. Further, securing the catheter to the retainer can include wrapping the catheter around an arcuate surface within the retainer.

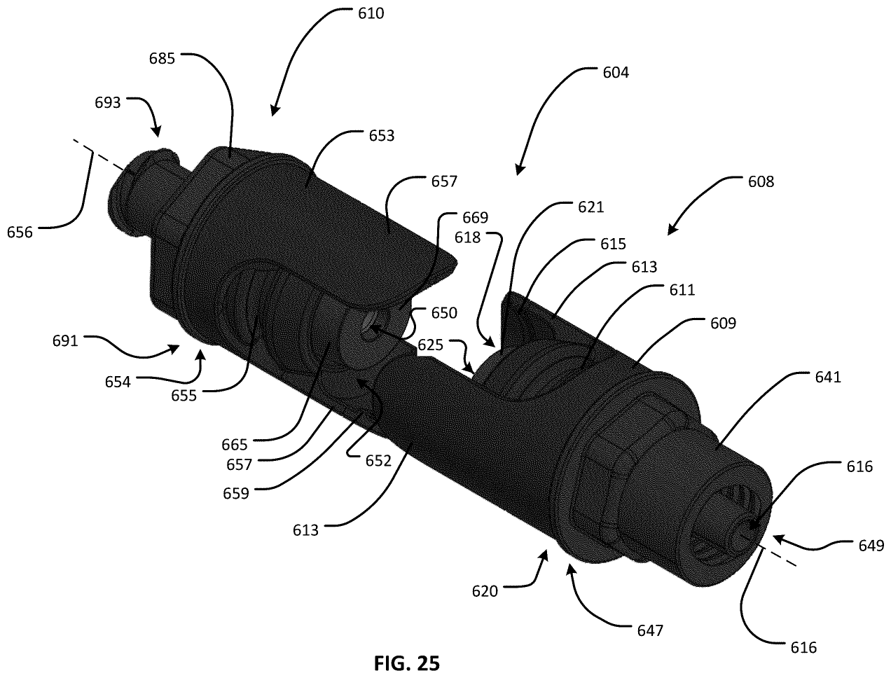

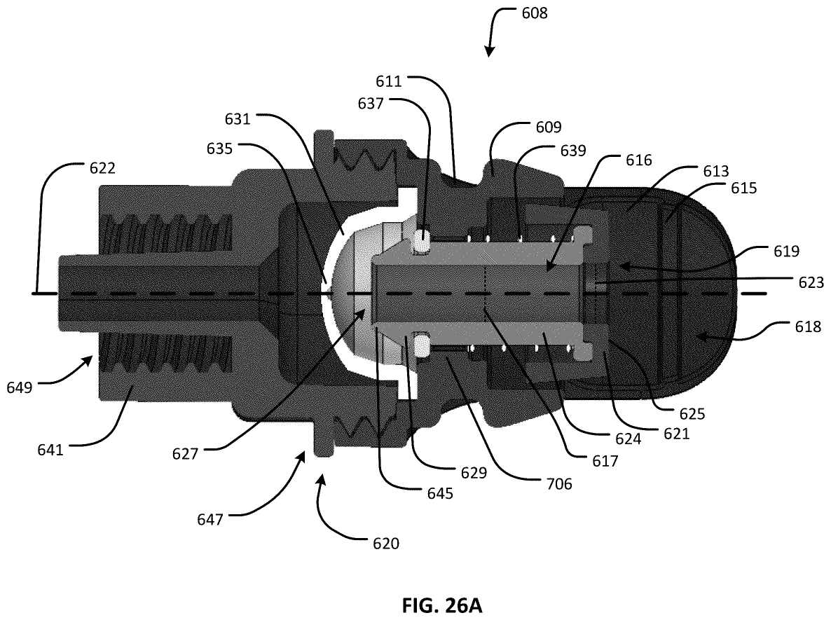

Another example of a failure-release 604 according to the present disclosure is shown in FIGS. 25-28. The failure-release 604 is similar to the failure-release coupling 104 described above and similar numbering in the 600 series is used for the failure-release 604. As will be described, like the above-described systems, a failure-release is provided to automatically disconnect the fluid connection between a first catheter and a second catheter upon the system receiving a force at or above a predetermined threshold to protect the percutaneous opening in the patient from the force. However, as will be described, the failure-release 604 may be adapted to be free of components, such as ferromagnetic materials and/or may utilize a mechanical coupling instead of a magnetic coupling. In this way the failure-release 604 may be safe to use with imaging or other applications, such s magnetic resonance imaging (MRI).

The failure-release 604 includes a first coupling 608 and a second coupling 610. The first coupling 608 includes a first coupling passageway 616 (e.g., passageway), a first end 618, and a second end 620. Similarly, the second coupling 610 includes a second coupling passageway 650 (e.g., passageway), a first end 652, and a second end 654. The first coupling passageway 616 defines a first coupling axis 622, and the second coupling passageway 650 defines a second coupling axis 656. In a non-limiting example, the first coupling is configured to connect to the first catheter 26. In a non-limiting example, the second coupling 610 is configured to connect to a second catheter. The first coupling 608 may include a first coupling body 609 and a Luer lock adapter 641. The Luer lock adapter 641 may be configured to be removably coupled to the first coupling body 609, as discussed in greater detail below. Other adapters or connections may be used instead of a Luer lock adapter 641.

The first coupling body 609 includes a recess 611 (e.g., a first coupling recess, a first recess), arms 613, and a piston 624. The arms 613 include a retention protrusion 615 (e.g., first coupling retention protrusion, first retention protrusion) at the first end 618 of the first coupling 608. The piston 624 includes a first piston end 619. At the first piston end 619, the piston 624 includes a piston fitting 621 (e.g., a first end fitting, a grommet, a seal, a suction seal, etc.). The piston fitting 621 includes a radial surface 625. The piston fitting 621 also defines a second inner diameter 623.

The piston 624 also includes a second piston end 627. Between the first piston end 619 and the second piston end 627, the piston 624 includes a first inner diameter 617. The first inner diameter 617 defines a diameter of the first coupling passageway 616 along the first coupling axis 622 inside the piston 624. In a non-limiting example, the first coupling axis is centered along the piston 624. The second inner diameter 623 defines a diameter of the first coupling passageway 616 along the first coupling axis 622 inside the piston fitting 621. In a non-limiting example, the first inner diameter 617 is larger than the second inner diameter 623. In a non-limiting example, the second inner diameter 623 is larger than the first inner diameter 617. In a non-limiting example, the second inner diameter 623 may be the same as the first inner diameter 617.

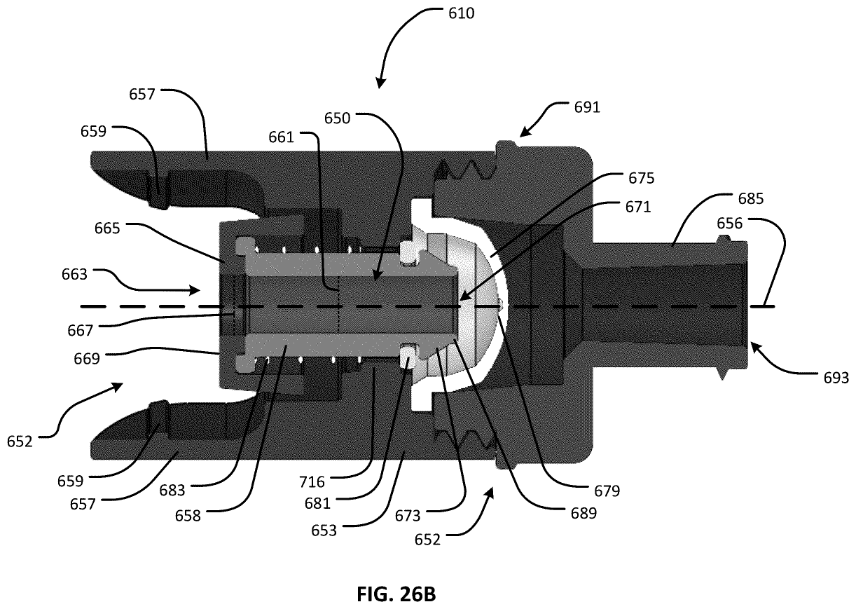

At the second piston end 627, the piston 624 includes a catch 629. In some examples, the catches 629 circumscribe an outside of the piston 624. In other examples, the catches 629 are a plurality of catches (e.g., a plurality of ribs, tabs, etc.) that circumscribe the outside of the piston 624. The catch 629, in a disengaged configuration (e.g., uncoupled, unlocked, disconnected, etc.) shown in FIG. 25 (and in FIGS. 26A and 26B), is between a valve seal 635 and an annular protrusion 706 of the first coupling body 609. In some non-limiting examples, a bottom of the catch 629, or a lip 645, is contacting, or close to contacting, the valve seal 635. In other non-limiting examples, a top of the catch 629 is contacting, or close to contacting, the annular protrusion 706 in the disengaged configuration.

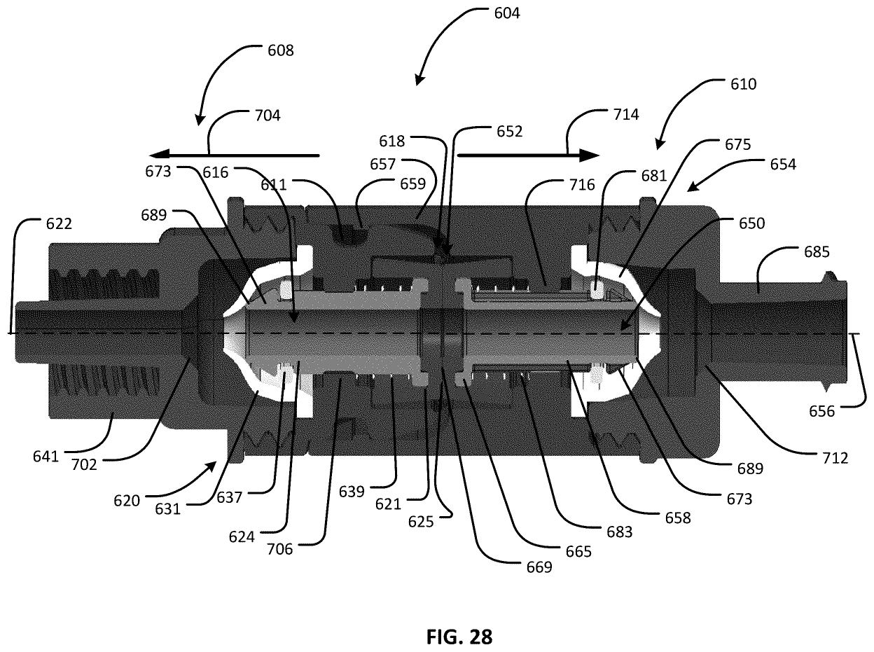

In a non-limiting example, the first coupling 608 includes a washer 637, as shown in FIGS. 26A, 26B, and 28. In some aspects, the washer 637 may contact, or be close to contacting, the top of the catch 629 in the disengaged configuration. In some non-limiting examples, the washer 637 may be coupled to the top of the catch 629.

A valve 631 (e.g., a first coupling valve, a first valve) is located near the second piston end 627 of the piston 624. In a non-limiting example, the valve 631 is a one-way valve. In a non-limiting examples, the valve 631 is configured to allow flow of a fluid from the first end 618 to the second end 620 of the first coupling 608. In a non-limiting example, the valve 631 is located near the second end 620 of the first coupling 608. The valve 631 includes, at an end proximate the second end 620 of the first coupling 608, the valve seal 635. At an end opposite the valve seal 635, the valve 631 includes the washer 637. In a non-limiting example, the washer 637 is inset into the valve 631.

In a non-limiting example, the valve 631 is secured in the first coupling 608 via the Luer lock adapter 641. When the Luer lock adapter 641 is secured within the first coupling 608, the valve 631 may be secured within the first coupling 608. When the Luer lock adapter 641 is secured to the first coupling 608, the valve 631 and the Luer lock adapter 641 create a seal at a first end 647 of the Luer lock adapter 641. In some aspects, the seal does not allow any fluid to leave the first coupling passageway 616.

The first coupling body 609 also includes a biasing component 639, e.g., a spring. The biasing component 639 may be positioned between the top of the catch 673 and the annular protrusion 706. In some non-limiting examples, the biasing component 639 is between the washer 637 and the valve seal 635. When the biasing component 639 is compressed or extended, the biasing component 639 creates a biasing force.

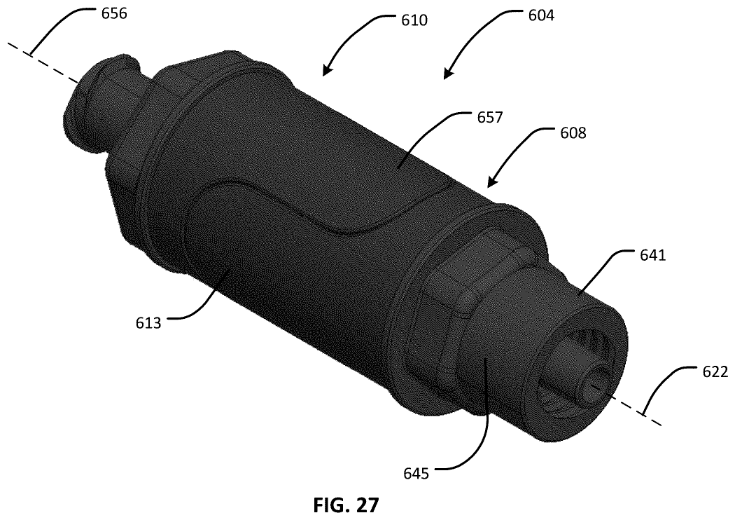

As mentioned above, the Luer lock adapter 641 is secured within the first coupling 608. In particular, the first end 647 the Luer lock adapter 641 is secured at the second end 620 of the first coupling 608. In a non-limiting example, the Luer lock adapter 641 is secured within the first coupling 608 via threads, where the Luer lock adapter 641 threads into the first coupling 608. In another non-limiting example, the Luer lock adapter 641 snap-fits into the first coupling 608. At a second end 649 of the Luer lock adapter 641, the Luer lock adapter 641 is configured to secure the first catheter 26. In the non-limiting example shown in FIGS. 27-28, the Luer lock adapter 641 is configured to be a female Luer lock adapter at the second end 649, e.g., the first catheter 26 is inserted into the Luer lock adapter 641. In other non-limiting examples, the Luer lock adapter 641 is configured to be a male Luer lock adapter.

The second coupling 610 includes substantially similar components as the first coupling 608, and, thus, the above discussion of the first coupling 608 applies to the second coupling 610. For example, referring to FIGS. 25-28, the second coupling 610 includes a second coupling body 653 and a Luer lock adapter 685. Similar to the first coupling 608, the Luer lock adapter 685 of the second coupling 610 includes a first end 691, and a second end 693. The Luer lock adapter 685 also includes a seal between Luer lock adapter 685 and a valve 675. In the non-limiting example shown in FIGS. 25-28, the Luer lock adapter 685 is configured to be a male Luer lock adapter, e.g., the second end 693 of the Luer lock adapter 685 is configured to be inserted into the second catheter 28. In other non-limiting examples, the Luer lock adapter 685 is configured to be a female Luer lock adapter.

Similar to the first coupling body 609, the second coupling body 653 includes a recess 655 (e.g., a second coupling recess, a second recess, arms 657 that include a retention protrusion 659 (e.g., second coupling retention protrusion, second retention protrusion); a piston 658 that includes a first inner diameter 661, a first piston end 663, and second piston end 671, a washer 681; a biasing component 683; an annular protrusion 716; and the valve 675 (e.g., a second coupling valve, a second valve) that includes a valve seal 679. In a non-limiting example, the valve 675 is a one-way valve. In a non-limiting examples, the valve 675 is configured to allow flow of a fluid from the first end 652 to the second end 654 of the second coupling 610. The first piston end 663 of the second piston 658 includes a piston fitting 665. The piston fitting 665 includes an inner diameter 667 and a radial surface 669. The second piston end 671 of the second coupling piston 658 includes a catch 673, and the catches include a lip 689. In some non-limiting examples, the second coupling body 653 is substantially identical to the first coupling body 609.

In a non-limiting example, the valve 675 is secured in the second coupling 610 via the Luer lock adapter 685. When the Luer lock adapter 685 is secured within the second coupling 610, the valve 675 may be secured within the second coupling 610. When the Luer lock adapter 685 is secured to the second coupling 610, the valve 675 and the Luer lock adapter 685 create a scal 687 at a first end 691 of the Luer lock adapter 685. In some aspects, the seal 687 does not allow any fluid to leave the second coupling passageway 650. In a non-limiting example, the second coupling 610 is configured to connect the second catheter 28 to fluidly connect the first catheter 26.

The failure-release 604 includes the retention protrusions 615 and recesses 611 of the first coupling 608 and the retention protrusions 659 and recesses 655 of the second coupling 610. In a non-limiting example, the failure-release 604 includes the first coupling body 609 and the second coupling body 653. In a non-limiting example, the failure-release 604 includes the first coupling 608 and the second coupling 610.

In the disengaged configuration shown in FIG. 25, the piston 624 of the first coupling 608, and, similarly, the piston 658 of the second coupling 610, are in a disengaged configuration. In a non-limiting example, the washer 637 and the catch 629 may contact the annular protrusion 706 of the first coupling 608. Similarly, in a non-limiting example, the washer 681 and the catch 673 may contact the annular protrusion 716 of the second coupling 610.

In the disengaged configuration of the failure-release 604, the valve seal 635 is uninterrupted, so no fluid can flow through the valve 631 in the first coupling passageway 616 along the first coupling axis 622. The piston 624 is spaced from the valve 631, and thus, the valve seal 635. Since the piston is spaced from the valve 631, the valve 631 is in a normal state (e.g., closed, closed configuration). Fluid may flow through the second end 649 to the first end 647 of the Luer lock adapter 641, but the fluid cannot flow through the valve 631, nor out the first coupling body 609 due to the seal between the Luer lock adapter 641 and the valve 631. Since the first and second couplings 608, 610 are substantially similar, the above description, as applied to the first coupling 608, applies to the second coupling 610.

In the disengaged configuration of the failure-release 604, the piston fitting 621 is spaced from the piston fitting 665. In particular, the radial surface 625 is spaced from the radial surface 669, e.g., the radial surfaces are not in contact.

In an engaged configuration (e.g., coupled, locked, connected, etc.) of the failure-release 604 shown in FIG. 27 and FIG. 28, the piston 624 of the first coupling 608, and, similarly, the piston 658 of the second coupling 610, are in an engaged configuration. In the engaged configuration, the retention protrusions 659 on the arms 657 of the second coupling 610 engage with the recess 611 of the first coupling 608. Further, the retention protrusions 615 on the arms 613 of the first coupling 608 engage with the recess 655 of the second coupling 610. When the first and second couplings 608, 610 are engaged, the radial surface 625 of the piston fitting 621 of the first coupling 608 is in contact with the radial surface 669 of the piston fitting 633 of the second coupling 610. Further, when the radial surfaces 625, 669 are in contact, or are fluidly connected, and as the first and second couplings 608, 610 are moved to the engaged configuration, the respective pistons of each coupling translate in an engagement direction toward the respective second end along the respective coupling axis. For example, when the failure release coupling 600 is in the engaged configuration, the first piston 624 translates along the first coupling axis 622 towards the second end 620 of the first coupling 608 in an engagement direction 704. The engagement direction 704 extends from the first end 618 to the second end 620. In a non-limiting example, the engagement direction 704 is parallel to the first coupling axis 622.

Continuing, as the first piston 624 translates along the engagement direction 704, the biasing component 639 is resiliently deflected, e.g., compressed. When the retention protrusions 615 of the first coupling body 609 are engaged with the recess 655 of the second coupling body 653, and vice versa (e.g., the retention protrusions 659 of the second coupling body 653 are engaged with the recess 611 of the first coupling body 609), the first piston 624 stops translating along the engagement direction 704.

Once the first piston 624 is in an engaged configuration, the catch 629 of the piston 624 protrudes through (e.g., interrupts) the valve seal 635. Specifically, the lip 645 of the catch 629 protrudes through the valve seal 635, allowing fluid to flow through the first coupling passageway 616 of the first coupling 608. In a non-limiting example, the lip 645 contacts an annular recess 702 of the Luer locking adapter 641, sealing the first coupling passageway 616 between the Luer locking adapter and the valve 631. The piston 624 is no longer spaced from the valve 631, nor the valve seal 635. Since the piston 624 is no longer spaced from the valve 631, the valve 631 is in an open state (e.g., open configuration).

The second piston 658, similar to the first piston 624, travels along an engagement direction 714, which extends from the first end 652 to the second end 654 and is opposite the engagement direction 704. Similar to the biasing component 639 of the first coupling 608, as the second piston 658 translates along the engagement direction 714, the biasing component 683 is resiliently deflected, e.g., compressed. When the biasing component 683 is resiliently deflected, the biasing component 683 creates a resilient force (e.g., spring force). When the retention protrusions 659 of the second coupling body 653 are engaged with the recess 611 of the first coupling body 609, and vice versa (e.g., the retention protrusions 615 of the first coupling body 609 are engaged with the recess 655 of the second coupling body 653), the second piston 658 stops translating along the engagement direction 714.

Once the second piston 658 is in an engaged configuration, similar to the first piston 624, the catch 673 protrudes through the valve seal 679. Specifically, the lip 689 of the catch 673 protrudes through the valve seal 679, allowing fluid to flow through the second coupling passageway 650 of the second coupling 610. In a non-limiting example, the lip 689 contacts an annular recess 712 of the Luer locking adapter 685, sealing the second coupling passageway 650 between the Luer locking adapter 685 and the valve 675. The piston 658 is no longer spaced from the valve 675, nor the valve seal 679. Since the piston 658 is no longer spaced from the valve 675, the valve 675 is in an open state (e.g., open configuration).

In the engaged configuration, fluid can flow from the first catheter 26 into the second end 649 of the Luer lock adapter 641 of the first coupling 608, along the first coupling axis 622, through the first coupling passageway 616 into the second coupling passageway 650, along the second coupling axis 656, and out the second end 693 of the Luer lock adapter 685 of the second coupling 610, and into the second catheter 28.

In the engaged configuration, the retention protrusions 615, 659 and corresponding recesses 611, 655 are configured to be a friction fitting. The failure-release 604 is configured to automatically disconnect the first coupling 608 from the second coupling 610, or vice versa (e.g., the second coupling is automatically disconnected from the first coupling) when a predetermined threshold is reached. In a non-limiting example, the failure-release 604 is configured to automatically disconnect the first coupling from the second coupling if a friction force has been overcome (e.g., the friction fitting has separated). In a non-limiting example, the predetermined threshold is the friction force of the friction fitting. In a non-limiting example, the friction force (e.g., engagement force) is greater than 10 Newtons. In another non-limiting example, the engagement force is greater than a weight of the drainage bag. In another non-limiting example, the engagement force is 5% greater than the weight of the drainage bag. In another non-limiting example, the engagement force is between 105-110%, 110-115%, 115-120%, 120-125%, 125-130%, 130-135%, 135-140%, 140-145%, 145-150%, or greater than 150% of the weight of the drainage bag. In another non-limiting example, the engagement force is greater 105% of the weight of the drainage bag and the resilient force of the biasing components 639. In other non-limiting examples, other engagement forces are considered.

In the engaged configuration of the failure-release 604, the piston fitting 621 is no longer spaced from the piston fitting 665, but, rather, the piston fitting 621 and the piston fitting 665 are in contact. In particular, the radial surface 625 is in contact with the radial surface 669.

In some non-limiting examples, the failure-release 604 may include a plurality of passageways 616, 650 to connect more than one catheter to each or either of the first coupling 608 and/or the second coupling 610. Correspondingly, in some examples, the first coupling body 609 may include a plurality of pistons 624 and/or the second coupling body 653 may include a plurality of pistons 658. In a non-limiting example, the first coupling body 609 may include two pistons 624. One of the pistons 624 can be configured to pass fluid. In one non-limiting example, the other piston 624 may be configured to pass gas (e.g., air). For example, in a non-limiting application, as fluid is drained (e.g., removed) via a first catheter 26, there may be a need to replace the volume of the fluid that is drained with a gaseous substance (e.g., air). Such a configuration may be desirable for clinical applications, such as chest tubes. In such non-limiting applications, it may be advantageous to include the plurality of pistons 624, and, in some aspects, include a plurality of pistons 658. Other variations or the inclusion of other numbers of components may be utilized as desirable for particular clinical applications.

Thus, while the invention has been described above in connection with particular configurations and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein.

We claim:

1. A system configured to be coupled to a percutaneous device, the system comprising:
a first coupling configured to connect to a first catheter extending through a percutaneous opening in a patient;
a second coupling configured to connect to a second catheter to fluidly connect to the first catheter; and
a failure-release configured to automatically disconnect the fluid connection between the first catheter and the second catheter upon the system receiving a force at or above a predetermined threshold to protect the percutaneous opening in the patient from the force.

2. The system of claim 1, wherein the failure-release includes a set of magnets, including a first magnet attached to the first coupling and a second magnet attached to the second coupling, the first and second magnets are removably coupled via a magnetic attraction therebetween.

3. The system of claim 2, wherein the predetermined threshold is equal to a magnetic attraction threshold of the magnetic attraction between the first and second magnets.

4. The system of claim 2, wherein the first and second magnets are ring magnets.

5. The system of claim 1, further comprising:
a retainer configured to be secured to skin of the patient adjacent the insertion site and configured to retain the first catheter therein.

6. The system of claim 5, wherein the retainer is sized to accommodate and configured to retain a portion of the first catheter formed in a coil.

7. The system of claim 6, wherein the retainer includes an arcuate surface around which the first catheter can be wrapped.

8. The system of claim 7, wherein the arcuate surface is textured to increase surface friction between the first catheter and the arcuate surface.

9. The system of claim 6, wherein the retainer includes a plurality of notches about a peripheral wall thereof through which the first catheter can pass through.

10. The system of claim 1, wherein the first coupling includes a one-way valve and the second coupling includes a stem configured to extend into the first coupling to open the one-way valve.

11. The system of claim 1 wherein the first coupling further includes a recess and retention protrusions, and wherein the second coupling includes a recess and retention protrusions.

12. The system of claim 11, wherein the failure-release includes the retention protrusions of the first coupling being secured to the recess of the second coupling, and wherein the failure-release includes the retention protrusions of the second coupling being secured to the recess of the first coupling.

13. The system of claim 1, wherein the predetermined threshold is equal to a friction force between the first and second couplings.

14. The system of claim 13, further comprising a first valve and a second valve, wherein the first coupling includes the first valve and the second coupling includes a second valve.

15. The system of claim 14, wherein the second catheter is fluidly connected to the first catheter when pistons of the each of the first and second couplings are pressed in a respective engagement direction.

16. The system of claim 1, wherein the system is free of ferromagnetic materials.

17. A system configured to be coupled to a percutaneous device, the system comprising:
a failure-release operable between an engaged configuration and a disengaged configuration; and
a one-way valve;
the failure-release and the one-way valve configured to be integrated with a catheter extending through a percutaneous opening in a patient;
with the failure-release in the engaged configuration the one-way valve is in an open configuration and fluid flow is permitted through the catheter, the one-way valve, and out through the failure-release;
with the failure-release in the disengaged configuration the one-way valve is in a closed configuration and fluid flow is prohibited through the catheter.

18. The system of claim 17, wherein the failure-release includes a set of magnets, including a first magnet and a second magnet;
with the failure-release in the engaged configuration, the first and second magnets are magnetically coupled;
with the failure-release in the disengaged configuration, the first and second magnets are magnetically decoupled and spaced apart.

19. The system of claim 18, wherein the failure-release includes a stem coupled to the second magnet and the one-way valve is coupled to the first magnet, with the failure-release in the engaged configuration, the stem urges the one-way valve out of a normally closed state, positioning the one-way valve in the open configuration.

20. The system of claim 19, wherein, with the failure-release in the disengaged configuration, the stem is spaced from the one-way vale, the one-way valve is in the normally closed state, positioning the one-way valve in the closed configuration.

21. The system of claim 17, further including a retainer configured to retain the catheter on the patient between the percutaneous opening and the failure-release.

22. The system of claim 21, wherein the retainer includes a cover and a base, the base being configured to be adhered to the patient.

23. The system of claim 22, wherein the cover or the base has an arcuate surface around which the catheter can be wrapped.

24. The system of claim 22, wherein the cover or the base has a plurality of notches about a peripheral wall thereof through which the catheter can pass through.

25. The system of claim 17, wherein the failure-release includes a recess and a retention protrusion that are coupled when the failure release is in the engaged configuration and are decoupled when the failure release is in the disengaged configuration.

26. The system of claim 25, wherein the failure-release includes a piston and, with the failure-release in the engaged configuration, the piston urges the one-way valve out of a normally closed state, positioning the one-way valve in the open configuration.

27. The system of claim 26, wherein, with the failure-release in the disengaged configuration, the piston is spaced from the one-way valve, the one-way valve is in the normally closed state, positioning the one-way valve in the closed configuration.

28. The system of claim 27, wherein the piston includes a fitting spaced on a radial surface when the failure-release in the disengaged configuration, and the fitting in contact with a radial surface, sealing the fluid flow within the system when the failure-release in the engaged configuration.

29. The system of claim 17, wherein the system is free of ferromagnetic materials.

30. A system configured to be used with a percutaneous device, the system comprising:
a retainer including a base, the base configured to be secured to skin of the patient adjacent a percutaneous opening in a patient; and
the retainer configured to retain a catheter extending through the percutaneous opening.

31. The system of claim 30, further comprising:
a first coupling configured to connect to a first catheter extending through a percutaneous opening in a patient;
a second coupling configured to connect to a second catheter, the second coupling configured to be coupled with the first coupling to fluidly connect to the first catheter and the second catheter; and
a failure-release configured to automatically disconnect the fluid connection between the first catheter and the second catheter upon the system receiving a force above a predetermined threshold to protect the percutaneous opening in the patient from the force.

32. The system of claim 31, wherein the first coupling and the second coupling are free of ferromagnetic materials.
